(19) United States
(12) Patent Application Publication
(10) Pub. No.: US 2016/0303151 A1
(43) Pub. Date: Oct. 20, 2016

(54) Topical Composition of Ivermectin

(71) Applicant: GAVIS PHARMACEUTICALS, Somerset, NJ (US)

(72) Inventor: Bala Chandran Nayar, Somerset, NJ (US)

(73) Assignee: GAVIS PHARMACEUTICALS, Somerset, NJ (US)

(21) Appl. No.: 14/687,750

(22) Filed: Apr. 15, 2015

(51) Int. Cl. A61K 31/7048; A61K 47/44; A61K 47/10; A61K 47/14; A61K 9/00; A61K 9/107

(52) U.S. Cl. CPC A61K 31/7048; A61K 9/0014; A61K 9/107; A61K 47/10; A61K 47/14; A61K 47/44

(57) ABSTRACT

A topical water-in-oil composition of ivermectin is provided. The topical water-in-oil composition contains an oily phase, an aqueous phase and a surfactant emulsifier. The emulsion is essentially devoid of any glycols.

### BACKGROUND OF THE INVENTION

(a) Field of the Invention

The present invention is directed to a topical composition of ivermectin. The composition is particularly in the form of a water-in-oil emulsion and comprises an aqueous phase dispersed in an oily phase which comprises fatty substances. The invention is also directed to use of said topical composition of ivermectin for the treatment of rosacea, and particularly, for the treatment of inflammatory lesions of rosacea.

(b) Description of the Related Art

Ivermectin is a mixture of two compounds belonging to the class of avermectins, 5-O-demethyl-22,23-dihydroavermectin A₁ₐ and 5-O-demethyl-22,23-dihydroavermectin A_{1b}. They are also known as 22,23-dihydroavermectin B₁ₐ and 22,23-dihydroavermectin B_{1b}. Ivermectin is a mixture containing at least 90% 5-O-demethyl-22,23-dihydroaverrnectin A₁ₐ and less than 10% 5-O-demethyl-25-de(1-methylpropyl)-22,23-dihyro-25-(1-methylethyl) avermectin A₁ₐ, generally referred to as 22,23-dihydro avermectin B₁ₐ and B_{1b}, or H₂B₁ₐ and H₂B_{1b}, respectively. Ivermectin contains at least 80% of 22,23-dihydroavermectin B₁ₐ and less than 20% of 22,23-dihydroavermectin B_{1b}. The structural formulas are:

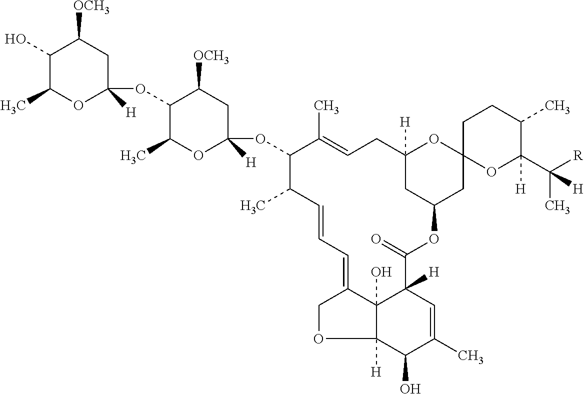

Component B₁ₐ R═C₂H₅ and Component B_{1b} R═CH₃

This active agent forms part of the class of avermectins, a group of macrocyclic lactones produced by the bacterium Streptomyces avermitilis (Reynolds J E F (Ed) (1993) Martindale, The Extra Pharmacopoeia. 29th Edition. Pharmaceutical Press, London). Avermectins include in particular ivermectin, invermectin, avermectin, abamectin, doramectin, eprinomectin and selamectin.

In the middle of the 1980s, ivermectin was presented as a broad-spectrum anti-parasitic medicinal product for veterinary use (W. C. Campbell, et al., (1983). Ivermectin: a potent new anti-parasitic agent, Science, 221, 823-828). It is effective against most common intestinal worms (except tapeworms), most acarids and some lice. It in particular exhibits considerable affinity for the glutamate-dependent chloride channels present in invertebrate nerve cells and muscle cells. Its binding to these channels promotes an increase in membrane permeability to chloride ions, resulting in hyperpolarization of the nerve or muscle cell. Neuromuscular paralysis which can lead to the death of certain parasites results therefrom. Ivermectin also interacts with other ligand-dependent chloride channels, such as those involving the neuromediator GABA (gamma-aminobutyric acid).

Ivermectin is more particularly an anthelmintic. It has been administered in humans in the treatment of onchocerciasis caused by onchocerca volvulus, of gastrointestinal strongyloidiasis (anguillulosis) (product Stomectol®) and of human scabies (Meinking T. L. et al., N. Engl. J. Med., 1995 Jul. 6; 333(1): 26-30, The treatment of scabies with ivermectin) and also in the treatment of microfilaremia diagnosed or suspected in individuals suffering from lymphatic filariosis due to Wuchereria bancrofti.

U.S. Pat. No. 4,199,569, discloses ivermectin as a semisynthetic, anthelmintic agent derived from the avermectins, a class of highly active broad-spectrum anti-parasitic agents isolated from the fermentation products of Streptomyces avermitilis.

U.S. Pat. Nos. 6,399,652; 6,399,651 and 6,319,945 disclose methods of treating skin disorders via application of topical formulations containing ivermectin to treat acne vulgaris (the '652 patent), a variety of dermatoses (e.g., transient acantholytic dermatitis, acne miliaris necrotica, acne varioliformis, perioral dermatitis, and acneiform eruptions; the '651 patent) and seborrheic dermatitis (the '945 patent).

U.S. Pat. No. 5,952,372 describes a method for treating rosacea using ivermectin orally or topically in order to reduce and eliminate the parasite Demodex folliculorum present on the skin of patients.

U.S. Pat. No. 6,133,310 discloses the use of ivermectin topically in the form of a prototype of a lotion consisting of a mixture of ivermectin and water, and also mentions the possibility of a prototype of a cream consisting, for its part, of a mixture of ivermectin and an excipient such as propylene glycol or sodium lauryl sulfate, but describes no pharmaceutical composition as such. Moreover, the elements disclosed in the patent provide no teaching to those skilled in the art regarding the feasibility of industrially acceptable pharmaceutical compositions containing ivermectin, in particular having good cosmetic properties and a shelf-life which is sufficiently long for an industrial pharmaceutical product (minimum of 2 years).

U.S. Pat. Nos. 7,550,440, 8,080,530, 8,093,219, 8,415,311, 8,470,788 and 8,815,816 disclose a topical pharmaceutical oil-in-water type emulsion of ivermectin comprising an oily phase, a surfactant-emulsifier, a mixture of solvent and/or pro-penetrating agents for ivermectin and gelling agents. A commercialized product of ivermectin based on these patents has recently been launched under the brand name Soolantra® by Galderma Laboratories. The product contains a large number of additives including those aforementioned, viz. carbomer copolymer type B, cetyl alcohol, citric acid monohydrate, dimethicone, edetate disodium, glycerin, isopropyl palmitate, methylparaben, oleyl alcohol, phenoxyethanol, polyoxyl 20 cetostearyl ether, propylene glycol, propylparaben, purified water, sodium hydroxide, sorbitan monostearate, and stearyl alcohol.

U.S. Pat. Appl. Pub. No. 2010/0093652 A1 discloses a topical formulation that inhibits the growth of microorganisms. The formulation comprises an effective amount of an insecticide dissolved in an oil phase comprising a water-miscible or water-soluble surface active agent, a suspending agent, and a non-ionic surfactant, and an aqueous phase comprising one or more preservatives, where the aqueous phase is buffered to a specific pH.

An inverse emulsion or water-in-oil emulsion containing an avermectin compound, notably ivermectin, was disclosed in U.S. Pat. No. 8,287,891. In the formulation a glycolic or aqueous/glycolic hydrophilic phase was dispersed in a continuous lipophilic phase. The formulation also contains an emulsifier having an HLB ranging from 2 to 7, and is useful for the treatment of a variety of dermatological conditions/afflictions, e.g., rosacea.

Chemical instability of ivermectin on contact with water is well known. In order to stabilize it, various solutions have been provided in the prior art.

European Pat. No. EP 0,045,655 B1 proposes forming micelles of surfactants which surround the ivermectin in order to protect it against water; other applications, such as PCT Application Pub. Nos. WO 01/60380 and WO 97/26895, propose using aqueous solvents for active agents, such as N-methyl-2-pyrrolidone. Finally, PCT Application Pub. Nos. WO 2004/093886 and WO 2005/089806 describe emulsions comprising an oily phase and an aqueous phase, said aqueous phase comprising a micellar active phase containing ivermectin.

The low compatibility of ivermectin with many excipients (N. O. Shaw, M. M. de Villiers and A. P. Lotter, Pharmazie, 54 (1999) 5, 372-376 Preformulation stability screening of ivermectin with non-ionic emulsion excipients), and its low solubility in water mean that pharmaceutical compositions containing ivermectin generally require either the addition of a large number of additives which make it possible to obtain stable compositions, which has the effect of increasing the risk of allergies.

Ivermectin is highly unstable in the presence of water and it proves to be particularly difficult to obtain stable topical compositions comprising ivermectin. It exhibits the difficulty of being very sparingly soluble and rarely stable in the cosmetic or pharmaceutical solvents commonly employed, in particular water; specifically, it is sensitive to an aqueous environment. This sensitivity to water can result in chemical instability of the active principle and/or in crystallization of the initially dissolved active principle. This sensitivity to water thus limits its formulation in cosmetic or dermatological compositions administered via the topical or oral route. In addition, by virtue of the low stability of ivermectin in water, the shelf life of aqueous compositions containing ivermectin is generally shorter than that of anhydrous compositions containing ivermectin.

The phenomena of chemical decomposition and/or of crystallization of ivermectin in the presence of water have as consequences a reduction in or loss of effectiveness and uncertainty with regard to the dose of active principle employed during the administration thereof, which militates against the desired objective. In addition, this decomposition of the active principle and/or its crystallization can modify the overall stability of the compositions and their appearance.

The pharmaceutical dosage form most commonly employed today in dermatology is the oil-in-water emulsion in which the active principle is preferably dissolved in the lipophilic phase. However, this solution remains rather unsatisfactory as, in order to meet an objective of concentration of active principle having a therapeutically quantifiable effectiveness, very high concentrations of solvating oils will be necessary, resulting in products which would without doubt be rather unpleasant to use due to their sticky feel, and being physically unstable, while remaining limited in the concentration of active principle.

One possibility is to dissolve the active principle in the hydrophilic phase of the emulsion, within the limit of its solubility in aqueous or aqueous/glycolic media.

However, this solution does not make it possible to solve the problems of chemical stability encountered with ivermectin as the activity in water of the emulsion remains very high.

The replacement of all or part of the aqueous phase by one or more glycols generally results in formulations which are not very acceptable cosmetically. In particular, above a level of 20% glycol, the formulation is not very acceptable cosmetically due to its sticky feel, and it is often not guaranteed to be physically stable.

There still exists a need to provide an alternative, chemically and physically stable and effective topical formulation of ivermectin which contains relatively fewer number of additives (or have minimum additive load), exhibit good physical and chemical stability and good tolerance on the skin.

### SUMMARY OF THE INVENTION

The present invention provides a topical water-in-oil emulsion composition of ivermectin. Particularly the topical water-in-oil emulsion composition of ivermectin comprises an oily phase, an aqueous phase and a surfactant emulsifier. Essentially, the emulsion is devoid of any glycols.

In one aspect, the invention provides a topical water-in-oil emulsion composition comprising:
(a) Ivermectin;
(b) an oily phase comprising fatty substances and emollients;
(c) a surfactant emulsifier; and
(d) water;
   wherein said emulsion is devoid of glycols.

In another aspect, the invention provides a topical water-in-oil emulsion composition comprising:
(a) ivermectin in an amount of about 0.01% to about 5% by weight;
(b) an oily phase comprising fatty substances in an amount of about 35% to about 45% by weight and emollients in an amount of about 5% to about 15% by weight;
(c) a surfactant emulsifier in an amount of about 5% to about 15% by weight; and
(d) water;
   wherein said emulsion is devoid of glycols.

In another aspect, the invention provides a topical water-in-oil emulsion composition comprising:
(a) ivermectin;
(b) an oily phase comprising fatty substances and emulsifiers selected from the group consisting of cetyl alcohol, stearyl alcohol, stearic acid, palmitostearic acid, self-emulsifiable wax, white petrolatum, mineral oil, soybean oil, isopropyl palmitate, 1-decene polymer (hydrogenated), C₁₂-C₁₅ alkyl benzoate, C₁₂-C₁₅ alkyl benzoates esters, lanolin alcohol and isopropyl myristate;
(c) a surfactant emulsifier selected from the group consisting of glyceryl/PEG100 stearate, sorbitan monostearate, sorbitan palmitate, Steareth-20, Steareth-2, Steareth-21, Ceteareth-20 and cetostearly alcohol; and
(d) water;
   wherein said emulsion is devoid of glycols.

In another aspect, the invention provides a topical water-in-oil emulsion composition consisting essentially of:
(a) ivermectin;
(b) an oily phase comprising white petrolatum, mineral oil, lanolin alcohol and isopropyl myristate;
(c) a surfactant emulsifier comprising cetostearly alcohol; and
(d) water;
   wherein said emulsion is devoid of glycols.

The topical water-in-oil emulsion composition of ivermectin of the present invention is preferably in the form of a cream.

In another aspect, the surfactant emulsifier in the topical water-in-oil emulsion composition of ivermectin of the invention is preferably a non-ionic surfactant emulsifier.

In another aspect, the topical water-in-oil emulsion composition of ivermectin of the invention remains chemically and/or physically stable over a period of time of at least 8 weeks.

In another aspect, the topical water-in-oil emulsion composition of ivermectin of the invention further comprises one or more additives selected from the group consisting of flavour enhancers, preserving agents, stabilizers, humidity regulators, pH regulators, osmotic pressure modifiers, UV-A screening agents, UV-B screening agents and antioxidants. Essentially, the topical water-in-oil emulsion composition of ivermectin of the invention is devoid of preserving agents.

In another aspect, the topical water-in-oil emulsion composition of ivermectin of the invention is devoid of a solvent for ivermectin, a propenetrating agent for ivermectin and a gelling agent.

In another aspect, the invention also provides a method for the treatment of rosacea, more particularly, for the treatment of inflammatory lesions of rosacea comprising topically applying the water-in-oil emulsion composition of ivermectin as substantially described herein above on to the affected skin of the patient in need of such treatment.

Still other aspects and advantages of the invention will be apparent from the following detailed description of the invention.

### DETAILED DESCRIPTION OF THE INVENTION

The invention provides for a topical water-in-oil emulsion composition of ivermectin with minimum additives load. It was surprisingly found that the composition according to the invention exhibits good stability, in particular at different pH, and good tolerance on the skin. Particularly, it was found that such stable compositions can be prepared without using glycols. Such formulations are also easy to manufacture on a commercial scale.

The ivermectin according to the invention may contain at least 80% of 22,23-dihydroavermectin B₁ₐ and less than 20% of 22,23-dihydroavermectin B_{1b}.

The topical water-in-oil emulsion compositions according to the invention are suited for treating the skin and may be in liquid, pasty or solid form, and more particularly in the form of ointments, creams, gels, sprays, foams, suspensions, lotions, shampoos or washing bases. The compositions may also be in the form of suspensions of microspheres or nanospheres or of lipid or polymeric vesicles or of polymeric patches and of hydrogels for controlled release. These compositions for topical application may be in anhydrous form, in aqueous form or in the form of an emulsion.

In a preferred embodiment of the invention, the composition is in the form of an emulsion of the cream or lotion type, of a gel, or of a solution, and more particularly in the form of cream.

Conventional emulsions known or described in the prior art are unstable, virtually homogeneous systems of two immiscible liquids, one of which is dispersed in the other in the form of fine droplets (micelles). This dispersion is stabilized by virtue of the action of surfactant-emulsifiers which modify the structure and the ratio of the forces at the interface, and therefore increase the stability of the dispersion by decreasing the interface tension energy.

Surfactant emulsifiers are amphiphilic compounds which possess a hydrophobic component having affinity for oil and a hydrophilic component having affinity for water, thus creating a link between the two phases. Ionic or nonionic emulsifiers therefore stabilize oil/water emulsions by adsorbing to the interface and forming lamellar layers of liquid crystals.

The compositions according to the invention particularly contain one or more non-ionic surfactant emulsifiers. The emulsifier power of non-ionic surfactants is closely linked to the polarity of the molecule. This polarity is defined by the HLB (hydrophilic/lipophilic balance). Conventional emulsions are generally stabilized by a mixture of surfactants, the HLBs of which can be quite different but the proportion of which in the mixture corresponds to the required HLB of the fatty phase to be emulsified.

Suitable non-ionic surfactant emulsifier can be selected from the group consisting of Cetostearyl alcohol, Cetyl alcohol, Cocamide DEA, Cocamide MEA, Isoceteth-20, Oleyl alcohol, Sorbitan monostearate, Sorbitan tristearate, Stearyl alcohol, tyloxapol, softigen, solutol HS15, poloxamers such as Pluronic F-68LF™ or Lutrol F68, Pluronic L-62LF™ and Pluronic L62D™ (BASF Wyandotte Corp., Parsippany, N.J., USA), polysorbates such as polysorbate 20 and polysorbate 80, polyoxyethylene fatty acid esters such as Emulphor™ (GAF Corp., Wayne, N.J., USA). The preferred surfactant emulsifier is Cetostearyl alcohol.

The compositions according to the invention are described as stable water-in-oil emulsions in that they may exhibit good physical and chemical stability over time, even at a temperature above ambient temperature (for example 45-55° C.).

The ivermectin in the composition according to the invention also, surprisingly, exhibits good chemical stability in the case of pH variation.

The compositions according to the invention are advantageously water-in-oil emulsions which comprise:
(a) Ivermectin;
(b) an oily phase comprising fatty substances and emollients;
(c) a surfactant emulsifier; and
(d) water.

Essentially the emulsion is devoid of glycols. In an embodiment, the emulsion is devoid of a solvent for ivermectin, a propenetrating agent for ivermectin and/or a gelling agent.

The oily phase of the composition according to the invention may comprise fatty substances, for example, vegetable, mineral, animal or synthetic oils, silicone oils, Guerbet alcohols or other substances, and mixtures thereof.

As an example of a mineral oil, mention may be made, for example, of paraffin oils of various viscosities, such as Primol 352, Marcol 82 or Marcol 152 marketed by Esso. As a vegetable oil, mention may be made of sweet almond oil, palm oil, soybean oil, sesame oil and sunflower oil. As an animal oil, mention may be made of lanolin, squalene, fish oil and mink oil. As a synthetic oil, mention may be made of esters, such as cetearyl isononanoate marketed in particular under the name Cetiol SN by Cognis France, diisopropyl adipate, for instance the product marketed under the name Ceraphyl 230 by ISF, isopropyl palmitate, for instance the product marketed under the name Crodamol IPP by Croda, or caprylic capric triglyceride such as Miglyol 812 marketed by Huls/Lambert Riviere. As a silicone oil, mention may be made of a dimethicone, such as the product marketed under the name Dow Corning 200 fluid, or a cyclomethicone, such as the product marketed under the name Dow Corning 244 fluid by Dow Corning, or the product marketed under the name Mirasil CM5 by SACI-CFPA.

Other fatty substances may include fatty acids such as white petrolatum, stearic acid, fatty alcohols such as stearyl alcohol, cetostearyl alcohol and cetyl alcohol, or derivatives thereof, waxes such as beeswax, carnauba wax or candelilla wax, and also gums, in particular silicone gums.

Fatty substances in the composition may be present in an amount of about 30% to about 45% by weight of the composition, more preferably in an amount of about 35% to about 45% by weight of the composition.

The oily phase of the composition according to the invention comprise emollients, for example, vegetable, mineral, animal or synthetic oils, silicone oils, isopropyl palmitate, 1-decene polymer (hydrogenated), C₁₂-C₁₅ alkyl benzoate, C₁₂-C₁₅ alkyl benzoates esters, lanolin alcohol and isopropyl myristate; and mixtures thereof.

Emollients in the composition may be present in an amount of about 5% to about 15% by weight of the composition.

In a preferred embodiment, the oily phase of the composition according to the invention comprises a mixture of fatty substances and emollients, preferably at least two emollients. The preferred fatty substances are white petrolatum and mineral oil and preferred emollients are lanolin alcohol and isopropyl myristate.

The oily phase of the emulsion according to the invention may be present at a content of from 15 to 45% by weight relative to the total weight of the composition, and preferably from 20 to 40% by weight.

The composition according to the invention advantageously comprises up to 15% by weight of a suitable surfactant emulsifier, preferably from about 5% to about 15% by weight of the composition.

The composition according to the invention comprises from 0.001 to 10% of ivermectin by weight relative to the total weight of the composition. Preferably, the composition according to the invention contains from 0.1 to 5% of ivermectin by weight relative to the total weight of the composition.

The composition of the invention also contains water ranging from 10 to 48%, and preferably from 20 to 45%, by weight relative to the total weight of the composition. The water used in the composition according to the invention will preferably be purified water.

The composition according to the invention may also contain inert additives or combinations of these additives, such as flavour enhancers; preservatives; stabilizers; humidity regulators; pH regulators; osmotic pressure modifiers; UV-A and UV-B screening agents; and antioxidants. Preferably, the topical water-in-oil emulsion composition of ivermectin of the invention is devoid of preserving agents.

These additives may be present in the composition at from 0.001 to 20% by weight relative to the total weight of the composition.

The compositions according to the invention are advantageously water-in-oil emulsions which comprise:
(a) ivermectin in an amount of about 0.01% to about 5% by weight;
(b) an oily phase comprising fatty substances in an amount of about 35% to about 45% by weight and emollients in an amount of about 5% to about 15% by weight;
(c) a surfactant emulsifier in an amount of about 5% to about 15% by weight; and
(d) water.

The pH of the compositions preferably ranges from 6.0 to 6.5. Verification of the natural pH of the mixture and possible correction with a solution of a neutralizing agent, and also the incorporation of the optional additives, may be carried out, according to their chemical nature, during one of the steps of the method of preparation, described above.

This invention also features formulation of the compositions according to the invention into pharmaceutical preparations useful to treat dermatological conditions/afflictions.

The formulation of ivermectin into topical water-in-oil emulsion compositions for human use according to the invention is particularly useful for the treatment of rosacea, of common acne, of seborrhoeic dermatitis, of perioral dermatitis, of acneform rashes, of transient acantholytic dermatosis, and of acne necrotica miliaris.

The formulation of ivermectin into topical water-in-oil emulsion compositions for human use according to the invention is more particularly useful in a regime or regimen for the treatment of rosacea.

### Example 1

### Ivermectin Cream

TABLE 1

| Sr. | | Quantity | Quantity | Quantity |
|---|---|---|---|---|
| No. | Ingredients | (% w/w) | (% w/w) | (% w/w) |
| 1 | Ivermectin | 1.00 | 2.00 | 3.00 |
| 2 | White Petrolatum | 30.00 | 30.00 | 30.00 |
| 3 | Lanolin Alcohol | 3.00 | 3.00 | 3.00 |
| 4 | Cetostearly Alcohol | 10.00 | 11.00 | 12.00 |
| 5 | Isopropyl Myristate | 8.00 | 8.00 | 8.00 |
| 6 | Heavy Mineral Oil | 9.00 | 9.00 | 9.00 |
| 9 | Purified Water | QS | QS | QS |

Process:

White Petrolatum was melted with heavy mineral oil to form a first mixture. Separately, lanolin alcohol, cetostearly alcohol and isopropyl myristate were melted together followed by addition of ivermectin and then mixed till dissolved to form a second mixture. The two mixtures then were mixed together and purified water was then added to the resulting mixture with homogenization to get a cream. The cream was then filled and packed into laminate/aluminum tubes.

What is claimed is:

1. A topical water-in-oil emulsion composition comprising:
(a) Ivermectin;
(b) an oily phase comprising fatty substances and emollients;
(c) a surfactant emulsifier; and
(d) water;
wherein said emulsion is devoid of glycols.

2. The composition of claim 1, wherein said composition is devoid of a solvent for ivermectin, a propenetrating agent for ivermectin and/or a gelling agent.

3. The composition of claim 1, wherein ivermectin is present in an amount of about 0.01% to about 5% by weight of said composition.

4. The composition of claim 1, wherein one or more fatty substances are present in an amount of about 35% to about 45% by weight of said composition.

5. The composition of claim 1, wherein one or more emollients are present in an amount of about 5% to about 15% by weight of said composition.

6. The composition of claim 1, wherein one or more surfactant emulsifiers are present in an amount of about 5% to about 15% by weight of said composition.

7. The composition of claim 1, wherein said composition is in the form of a cream.

8. The composition of claim 1, wherein said surfactant emulsifier is a non-ionic surfactant.

9. The composition of claim 8, wherein said non-ionic surfactant is selected from the group consisting of Cetostearyl alcohol, Cetyl alcohol, Cocamide DEA, Cocamide MEA, Isoceteth-20, Oleyl alcohol, Sorbitan monostearate, Sorbitan tristearate, Stearyl alcohol, tyloxapol, softigen, poloxamer, polysorbate 20, polysorbate 80, and polyoxyethylene fatty acid esters.

10. The composition of claim 1, wherein said composition remains chemically and/or physically stable over a period of time of at least 8 weeks.

11. The composition of claim 1, wherein said composition further comprises one or more additives selected from the group consisting of flavour enhancers, preserving agents, stabilizers, humidity regulators, pH regulators, osmotic pressure modifiers, UV-A screening agents, UV-B screening agents and antioxidants.

12. The composition of claim 1, wherein said composition is devoid of preserving agents.

13. A method for the treatment of rosacea or inflammatory lesions of rosacea comprising topically applying the composition of claim 1 on to the affected skin of the patient in need of such treatment.

14. A topical water-in-oil emulsion composition comprising:
(a) ivermectin;
(b) an oily phase comprising fatty substances and emulsifiers selected from the group consisting of cetyl alcohol, stearyl alcohol, stearic acid, palmitostearic acid, self-emulsifiable wax, white petrolatum, mineral oil, soybean oil, isopropyl palmitate, 1-decene polymer (hydrogenated), C₁₂-C₁₅ alkyl benzoate, C₁₂-C₁₅ alkyl benzoates esters, lanolin alcohol and isopropyl myristate;
(c) a surfactant emulsifier selected from the group consisting of glyceryl/PEG100 stearate, sorbitan monostearate, sorbitan palmitate, Steareth-20, Steareth-2, Steareth-21, Ceteareth-20 and cetostearly alcohol; and
(d) water;
wherein said emulsion is devoid of glycols.

15. A topical water-in-oil emulsion composition consisting essentially of:
(a) ivermectin;
(b) an oily phase comprising white petrolatum, mineral oil, lanolin alcohol and isopropyl myristate;
(c) a surfactant emulsifier comprising cetostearly alcohol; and
(d) water;
wherein said emulsion is devoid of glycols.

16. The topical water-in-oil emulsion composition of claim 15, wherein the compositions consists of:
(a) ivermectin;
(b) an oily phase consisting of white petrolatum, mineral oil, lanolin alcohol and isopropyl myristate;
(c) a surfactant emulsifier consisting of cetostearly alcohol; and
(d) water;
wherein said emulsion is devoid of glycols.

17. The topical water-in-oil emulsion composition of claim 15, wherein the compositions consists of:
(a) ivermectin present at about 1 to about 3% w/w;
(b) an oily phase consisting of white petrolatum present at about 20% w/w, mineral oil present at about 9% w/w, lanolin alcohol present at about 3% w/w and isopropyl myristate present at about 8% w/w;
(c) a surfactant emulsifier consisting of cetostearly alcohol present at about 10-12% w/w; and
(d) water;
wherein said emulsion is devoid of glycols.
